(19) United States
(12) Patent Application Publication
(10) Pub. No.: US 2025/0354171 A1
(43) Pub. Date: Nov. 20, 2025

(54) ERYTHROID-SPECIFIC PROMOTER AND METHOD OF USE THEREOF

(71) Applicant: St. Jude Children's Research Hospital, Inc., Memphis, TN (US)

(72) Inventors: Byoung Ryu, Memphis, TN (US); Matthew M. Wielgosz, Memphis, TN (US); Francesca Ferrara, Memphis, TN (US)

(21) Appl. No.: 19/285,342

(22) Filed: Jul. 30, 2025

Related U.S. Application Data
(63) Continuation of application No. 16/309,289, filed on Dec. 12, 2018, now abandoned.
(60) Provisional application No. 62/359,471, filed on Jul. 7, 2016.

(51) Int. Cl. C12N 15/86; A61K 48/00; C07K 14/47

(52) U.S. Cl. CPC C12N 15/86; C07K 14/47; A61K 48/00; C12N 2740/15043; C12N 2830/008

(57) ABSTRACT

A DNA construct containing an erythroid lineage-specific promoter operably linked to a nucleotide coding sequence of interest and a method of using the same in the prevention or treatment a hematopoietic disorder such as a hemoglobinopathy are described.

### INTRODUCTION

This application is a continuation of U.S. patent application Ser. No. 16/309,289 filed Dec. 12, 2018 which is the National Stage of PCT/US2017/040664 filed Jul. 5, 2017. This application claims the benefit of priority from U.S. Provisional Patent Application Ser. No. 62/359,471, filed Jul. 7, 2016, the content of which is incorporated herein by reference in its entirety.

### STATEMENT REGARDING ELECTRONIC FILING OF A SEQUENCE LISTING

A Sequence in XML Listing format, entitled SJ0071US_C1.xml, 22,547 bytes in size, generated on Jul. 30, 2025, is filed herewith. This Sequence Listing is hereby incorporated herein by reference into the specification for its disclosures.

### BACKGROUND

Recent progress in the field of gene therapy has indicated that patients afflicted with hemoglobinopathies such as β thalassemia or sickle cell anemia will benefit from novel therapeutic approaches. Transplantation of hematopoietic cells (HCs) modified with lentiviral vectors carrying the β-globin gene has resulted in long-term correction of several mouse models of hemoglobin disorders. Although infusion of genetically modified autologous cells avoids the risk of GVHD and immunosuppressive pretransplant conditioning and addresses the lack of compatible donors, this therapy faces at least three substantive caveats: the requirement for toxic myeloablation; current gene transfer methods are unable to transduce more than a fraction of hematopoietic stem (HSCs); and various in vivo selection strategies available suffer from suboptimal efficacy and safety. For example, β thalassemic recipient mice require at least 200 rads of irradiation and a very high dose of bone marrow cells (>20×10⁶) to achieve stable engraftment and phenotypic improvement. Accordingly, there is a need in the art for improved methods of gene therapy for the treatment or prevention of hematopoietic disorders.

### SUMMARY OF THE INVENTION

This invention is a DNA construct containing an erythroid lineage-specific promoter from a glycophorin A gene; and a nucleotide coding sequence operably linked to the promoter sequence. In certain embodiments, the erythroid-specific promoter comprises or consists of SEQ ID NO:1 or SEQ ID NO:2. In other embodiments, the DNA construct further includes an erythroid-specific enhancer, e.g., a BCL11A+58 enhancer, GATA-1 enhancer or HS40 enhancer. In further embodiments, the nucleotide coding sequence encodes a RNA or protein, e.g., an artificial zinc finger protein. In further embodiments, the construct is a gene therapy vector, e.g., a viral vector.

This invention is also a DNA construct containing an erythroid lineage-specific glycophorin A, Ankyrin, beta-Spectrin or Adducine 2 promoter; a BCL11A+58, GATA-1 or HS40 enhancer; and a nucleotide sequence encoding a BCL11A inhibitory RNA molecule operably linked to the promoter. In certain embodiments, the BCL11A inhibitory RNA molecule is an siRNA, shmiR, or shRNA and the construct is a gene therapy vector, e.g., a viral vector.

Methods for preventing or treating a hematopoietic disorder, e.g., a hemoglobinopathy, by administering a DNA construct to a subject in need of treatment is also provided.

### BRIEF DESCRIPTION OF THE DRAWINGS

FIG. 1 depicts lentiviral vectors containing the LCR β-globin (pGlobin), Ankyrin (pANK), Glycophorin A (pGPA), Spectrin (pSPEC), or Adducin2 (pADD2) promoter sequences were generated with eGFP as a reporter protein. CMV, CMV promoter; R, HIV-1 LTR R region; U5, HIV1 LTR U5 region; ψ, packaging signal; gag, nucleocapsid capsid; CPPT, central polypurine tract; RRE, rev responsive element; eGFP, enhanced green fluorescent protein; ΔU3, U3 region of the HIV-1 LTR in which a 400 bp deletion was introduced; pA, polyadenylated region.

FIG. 2 depicts the experimental protocol for accessing promoter expression levels.

### DETAILED DESCRIPTION OF THE INVENTION

A promoter for restricting expression of a nucleotide coding sequence of interest only to cells of the erythroid lineage has now been identified. This promoter, the glycophorin A promoter, is of particular use in gene therapy constructs, e.g., viral vectors, for the treatment and/or prevention of hematopoietic disorders such as hemoglobinopathies. The use of this cell lineage-specific expression control sequence offers safety advantages in restricting nucleic acid expression to this single lineage; and thus, DNA constructs of the invention alleviate concerns dealing with ectopic expression of nucleic acids in undesired cells types, can which lead toxicity and/or to immunogenicity. Accordingly, this invention provides a DNA construct harboring the erythroid lineage-specific promoter from glycophorin A and methods of using the same in the prevention and/or treatment of hematopoietic disorders.

A DNA construct of invention is intended to include any contiguous span of DNA harboring the erythroid lineage-specific promoter from glycophorin A of this invention operably linked to a nucleotide coding sequence of interest. The DNA construct can be a cloning vector, expression vector or expression cassette. As used herein, the term “cloning vector” refers to a circular DNA molecule minimally containing the erythroid lineage-specific glycophorin A promoter operably linked to a nucleotide coding sequence of interest, an Origin of Replication, a means for positive selection of host cells harboring the vector (e.g., an antibiotic-resistance gene), and a multiple cloning site. Cloning vectors are conventionally used for transfer of a nucleotide sequence of interest into an appropriate host cell for duplication during propagation of the host.

By comparison, the term “expression vector” means a nucleic acid that has the ability to confer expression of a nucleotide coding sequence of interest to which it is operably linked, in a cell or cell-free expression system. Within the context of the present invention, it is to be understood that an expression vector that comprises a promoter as defined herein may be a plasmid, bacteriophage, phagemid, cosmid, virus, or other nucleic acid capable of maintaining and or replicating heterologous DNA in an expressible format should it be introduced into a cell.

The term “expression cassette” refers to the part of an expression vector which encodes a nucleotide coding sequence of interest for expression by the expression vector. For example, the cassette refers to the part of the expression vector which includes the erythroid lineage-specific promoter from glycophorin A operably linked to a nucleotide coding sequence of interest. In a particular embodiment of the invention, the expression cassette contains the coding sequence of a therapeutic gene used to treat, prevent, or ameliorate a genetic disorder, such as a hematopoietic disorder. The cassette may further include other DNA sequences, such as enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, nucleotides encoding self-cleaving polypeptides or epitope tags.

The term “promoter,” as used herein, refers to a DNA region which contains an initial binding site for RNA polymerase and facilitates the transcription of a particular nucleotide coding sequence downstream thereof. That is, a promoter is an untranslated nucleotide sequence, upstream of a coding region, to which RNA polymerase binds to initiate the transcription of a nucleotide coding sequence, and is typically located near the nucleotide coding sequence it regulates, on the same strand and upstream (towards the 5′ region of the sense strand). Ideally, the promoter includes a nucleotide sequence capable of activating erythroid lineage-specific expression of operably linked nucleotide coding sequence and in some embodiments the nucleotide sequence will retain the minimum binding site(s) for transcription factor(s) required for the sequence to act as a promoter. In accordance with this invention, the erythroid lineage-specific promoter is from glycophorin A. The glycophorin A gene is known and located at positions 144119363-144140693 of human chromosome 4. Further, a DNA fragment covering −750 to +36 of the 5′-flanking sequence of glycophorin A gene, relative to the transcriptional start site, has been described. See Kudo, et al. (1994) J. Biochem. 116(1):183-92.

Conventionally, globin gene therapy vectors have used the locus control region (LCR) enhancer and beta-globin promoter to control expression of globin. In the context of viral vectors, this LCR-promoter is large (˜3.5 kb), which reduces viral vector titer. In contrast, the promoter of the present invention is between about 300 bp and 600 bp in length (including all intervening numbers). More preferably, the promoter of the present invention is about 550 bp, 500 bp, 450 bp or 400 bp in length. In particular embodiments, the erythroid-lineage specific promoter has a significant enrichment of the H3K27Ac mark (histone H3 lysine 27 acetylation), which is a known promoter mark (Wang, et al. (2008)Nat. Genet. 40(7):897-903). The H3K27Ac mark is often found near regulatory elements (7 cell lines), as well as DNAse I hypersensitivity peak clusters (95 cell types) from ENCODE (the Encyclopedia of DNA Elements project).

In some embodiments of this invention, the erythroid lineage-specific promoter from glycophorin A comprises or consists of the nucleotide sequence:

(SEQ ID NO: 1)
ttgcatctcatgtctcttacattgctgtgtggctcaccatgagtttggga
gtctttcagaacctcagaacactcaaatgatttaaatttctcaaatacat
tcatttcacatataggaagtcactttcatttggaccactgggtcttgaca
ttagaaatgagaaggtccatggctccacaacagctacctcagcctggcac
gtgccctggcctcagagattcacagtccagttctttgtccagttgggtgg
ctcctgtctaccaccttaccatgcccacttaactgatgcaaagttaatat
cacaagtagcaacctgttccttgcagtgaaaattttacttaccactttca
tagccccaagatatccatgtatctttattaacaggcgcttaacaacttgc
atcattt.

In other embodiments of this invention, the erythroid lineage-specific promoter from glycophorin A comprises or consists of the nucleotide sequence:

(SEQ ID NO: 2)
ttgcatctcatgtctcttacattgctgtgtggctcaccatgagtttggg
agtctttcagaacctcagaacactcaaatgatttaaatttctcaaatac
attcatttcacatataggaagtcactttcatttggaccactgggtcttg
acattagaaatgagaaggtccatggctccacaacagctacctcagcctg
gcacgtgccctggcctcagagattcacagtccagttctttgtccagttg
ggtggctcctgtctaccaccttaccatgcccacttaactgatgcaaagt
taatatcacaagtagcaacctgttccttgcagtgaaaattttacttacc
actttcatagccccaagatatccatgtatctttattaacaggcgcttaa
caacttgcatcatttaaaatgcctcccctgcctatcagctgatgatggc
cgcaggaaggtgggcctggaagataacagctagcaggctaaggtcagac
actgacacttgc.

The erythroid lineage-specific promoter from glycophorin A may also include allelic variants and derivatives (such as deletions, insertions, inversion, substitutions or addition of sequences) of this nucleotide other erythroid lineage-specific promoter sequence and sequences provided such variants or derivatives activate erythroid lineage-specific transcription of operably linked sequences. In various embodiments, such variants and derivatives are greater than 508, 80% to 100% at least 80%, at least 90% or at least 95% identical to SEQ ID NO:1 or SEQ ID NO:2.

The promoter or enhancer is considered erythroid lineage-specific when it provides for expression of a nucleotide coding sequence of interest only in a cell of the erythroid lineage. Cells of the erythroid lineage include, but are not limited to, e.g., proerythroblasts, basophilic erythroblasts, polychromatophilic erythroblasts, orthochromatic erythroblasts, polychromatophilic erythrocytes, and erythrocytes (red blood cells).

This invention also provides for the use of an erythroid-specific enhancer in combination with the glycophorin A promoter to increase promoter strength without affecting lineage expression. Erythroid-specific enhancers BCL11A+58 enhancer, GATA-1 enhancer and HS40 enhancer, in two different orientations, were analyzed in combination with the glycophorin A promoter or Ankyrin promoter. This analysis indicated that human GATA1 enhancer and BCL11A+58 erythroid specific enhancer provided the best enhancement of expression. Accordingly, the DNA constructs and methods of this invention can optionally include one or a combination of erythroid-specific enhancers in combination with the glycophorin A promoter. Ideally, the one or combination of erythroid-specific enhancers are provided upstream of the glycophorin A promoter (i.e., 5′ of the glycophorin A promoter).

In some embodiments of this invention, the erythroid-specific enhancer comprises or consists of the BCL11A+58

erythroid specific enhancer nucleotide
sequence:
(SEQ ID NO: 3)
gaggcccccctgggcaaacggccaccgatggagaggtctgccagtcctct
tctaccccacccacgcccccaccctaatcagaggccaaacccttcctgga
gcctgtgataaaagcaactgttagcttgcactagactagcttcaaagttg
tattgacc.
See Canver, et al. (2015) Nature 527:192-7.

In other embodiments of this invention, the erythroid-specific enhancer comprises or consists of the GATA-1 enhancer nucleotide sequence:

(SEQ ID NO : 4)
cctcgaggaatcatccctggctcccacctcagtttcccgcctccaaggca
gcatggcgggcaagaagttgaggccactgtccctgggtgttcctaccccc
acaccctcaccccaagacagcctgttactgcggcgccaacagccacggtc
gcctacatctgataagacttatctgctgccccagggcaggccggagctgg
cgtaagccccagt.
See Moreau-Gaudry, et al. (2000) Blood
98:2664-2672.

In still other embodiments of this invention, the erythroid-specific enhancer comprises or consists of the HS40 enhancer nucleotide sequence:

(SEQ ID NO: 5)
tctggaacctatcagggaccacagtcagccaggcaagcacatctgcccaa
gccaagggtggaggcatgcagctgtgggggtctgtgaaaacacttgaggg
agcagataactgggccaaccatgactcagtgcttctggaggccaacagga
cttctgagtcatcctgtgggggtggaggtgggacaagggaaaggggtgaa
tggtactgctgattacaacctctggtgctgcctccccctcctgtttatct
gagag.
See Moreau-Gaudry, et al. (2000) Blood
98:2664-2672.

A first nucleic acid sequence is operably linked with a second nucleic acid sequence when the sequences are placed in a functional relationship. For example, a nucleotide coding sequence is operably linked to a promoter if the promoter activates the transcription of the coding sequence. In this respect, an erythroid lineage-specific promoter from glycophorin A (and optionally an enhancer) is operably linked to a nucleotide coding sequence as it directs transcription of the nucleotide coding sequence.

The nucleotide coding sequence operably linked to the erythroid lineage-specific promoter from glycophorin A (and optionally an enhancer) may encode an RNA, e.g., an RNA such as an shmiR (i.e., a synthetic shRNA in miR backbone), miRNA, shRNA, antisense RNA or ribozyme; or a protein, e.g., a structural protein, an enzyme (e.g., nucleases such as ZEN and Crispr/Cas9 and artificial zinc fingers), transcription factor or a therapeutic protein. However, in accordance with this invention, the nucleotide coding sequence does not encode glycophorin A. Moreover, in certain embodiments, the nucleotide coding sequence does not encode a reporter or selectable marker. As is conventional in the art, a reporter or selectable marker is a protein/enzyme whose expression allows identification of cells that have been transformed with a DNA construct or vector containing the gene or polynucleotide. Selectable markers may provide resistance to toxic compounds such as antibiotics or herbicides, or provide detectable signals such as color or light.

In some embodiments, the nucleotide coding sequence of interest encodes a globin. The term “globin” is used herein to mean all proteins or protein subunits that are capable of covalently or noncovalently binding a heme moiety, and can therefore transport or store oxygen. Subunits of vertebrate and invertebrate hemoglobins, vertebrate and invertebrate myoglobins or mutants thereof are included by the term globin. Examples of globins include α-globin or a variant thereof, β-globin or a variant thereof, γ-globin or a variant thereof, and d-globin or a variant thereof. In one embodiment, the nucleotide coding sequence of interest is a nucleotide that encodes a polypeptide that provides a therapeutic function for the treatment of a hemoglobinopathy, e.g., α-globin, 3-globin or β-globin^{T87Q}. In certain embodiments, the nucleotide coding sequence is a wild type human β-globin gene, a deleted human β-globin gene comprising one or more deletions of intron sequences, or a mutated human β-globin gene encoding at least one anti-sickling amino acid residue. See U.S. Pat. Nos. 6,051,402, 5,861,488, 6,670,323, 5,864,029, and 5,877,288, which are herein incorporated by reference.

The role of stage-specific transcription factors in hemoglobin switching has been demonstrated (Sankaran, et al. (2008) Science 322(5909):1839-1842; Tanabe, et al. (2007) EMBO J. 26(9):2295-2306; Tanimoto, et al. (2000) Genes Dev. 14(21):2778-2794). In particular, the zinc finger transcription factor BCL11A has been shown to function as a repressor of HbF expression (Sankaran, et al. (2008) Science 322(5909):1839-1842). Further, when erythroid Krüppel-like factor 1 (EKLF1, KLF1), an adult β-globin gene-specific zinc finger transcription factor, was knocked down in erythroid progenitor CD34⁺ cells, γ-globin expression was induced (Zhou, et al. (2010) Nat. Genet. 42(9):742-744). DRED (direct repeat erythroid definitive) is a repressor complex that binds to the direct repeat (DR) elements in the ε- and γ-globin gene promoters, and two of the components in this complex are the orphan nuclear receptors TR2 and TR4 (Tanimoto, et al. (2000) Genes Dev. 14(21):2778-279). Enforced expression of TR2/TR4 increased fetal γ-globin gene expression in adult erythroid cells from β-YAC transgenic mice (Tanabe, et al. (2007) EMBO J. 26(9):2295-2306) and also in adult erythroid cells from the humanized SCD mice (Campbell, et al. (2011) Proc. Natl. Acad. Sci. USA 108:18808-18813). Accordingly, the manipulation of these transcription factors efficiently reactivates γ-globin expression during adult definitive erythropoiesis. In one embodiment, the expression of TR2 and/or TR4 is increased, e.g., by overexpression using a DNA construct of this invention. In another embodiment, the expression or activity of BCL11A and/or EKLF1 is reduced, e.g., by expressing an artificial zinc finger protein and/or an inhibitory RNA via a DNA construct of this invention.

Zinc finger proteins targeted to the proximal promoter of the ^{A}γ-globin gene have successfully induced γ-globin gene expression (Blau, et al. (2005) J. Biol. Chem. 280(44):36642-36647; Gräslund, et al. (2005) J. Biol. Chem. 280(5):3707-14; Tschulena, et al. (2011) Blood 117(10):2817-26; Wilber, et al. (2010) Blood 115 (15): 3033-3041). For example, the artificial zinc finger gg1-VP64 was designed to interact with the −117 region of the ^{A}γ-globin gene proximal promoter and provides a 7- to 16-fold increase in γ-globin expression in K562 cells stably transfected with gg1-VP64 (Gräslund, et al. (2005) J. Biol. Chem. 280(5):3707-14). Increased γ-globin gene expression was also observed following transfection of the gg1-VP64 construct into immortalized bone marrow cells isolated from human γ-globin locus yeast artificial chromosome (β-YAC) transgenic mice (Blau, et al. (2005) J. Biol. Chem. 280(44):36642-36647). Further, the gg1-VP64 activator was reported to significantly increase HbF levels in CD34⁺ erythroid progenitor cells from normal human donors and β-thalassemia patients (Wilber, et al. (2010) Blood 115(15):3033-3041; Wilber, et al. (2010) Blood 115(15):3033-3041).

Inhibitory RNA molecules are small RNAs that can bind to mRNA molecules and modulate activity, for example by preventing an mRNA from being translated into a protein. Inhibitory RNA include, but not limited to, siRNA, shmiR, miRNA, or shRNA. In one embodiment, the inhibitory RNA inhibits the expression of EKLF1. In another embodiment, the inhibitory RNA is a BCL11A inhibitory RNA that inhibits BCL11A expression thereby preventing hemoglobin switching. Inhibitory RNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. Preferably, the inhibitory RNA is identical to its target. The inhibitory RNA preferably targets only one sequence. Each of the RNA interfering agents, such as siRNAs, can be screened for potential off-target effects by, for example, expression profiling. Such methods are known to one skilled in the art and are described, for example, in Jackson, et al. (2003) Nature Biotechnology 6:635-637. In addition to expression profiling, one may also screen the potential target sequences for similar sequences in the sequence databases to identify potential sequences which may have off-target effects. For example, 15, or perhaps as few as 11 contiguous nucleotides, of sequence identity are sufficient to direct silencing of non-targeted transcripts. Therefore, one may initially screen inhibitory RNAs to avoid potential off-target silencing using the sequence identity analysis by any known sequence comparison methods, such as BLAST.

Exemplary inhibitory RNA molecules encoded by a DNA construct or viral vector of this invention and targeting BCL11A mRNA include, but are not limited to, siRNA molecules having the sequences of: GAGCACAAACGGAAACAAU (SEQ ID NO:6), GCCACAGGAUGACGAUUGU (SEQ ID NO:7), GCACUUAAGCAAACGGGA (SEQ ID NO:8), and ACAGAACACUCAUGGAUUA (SEQ ID NO:9); shRNA molecules having the sequence of: CCGGCGCACA GAACACTCAT GGATTCTCGA GAATCCATGA GTGTTCTGTG CGTTTT (SEQ ID NO:10), CCGGCCAGAG GATGACGATT GTTTACTCGA GTAAACAATC GTCATCCTCT GGTTTTTG (SEQ ID NO:11), GCGCTCGGAG ACTCCAGACA ACTCCATGTG GTAGAGTTGT CTGGAGTCTC CGAGCGC (SEQ ID NO:12), GCGCTTCTCT TGCAACACGC ACTCCATGTG GTAGAGTGCG TGTTGCAAGA GAAGCGC (SEQ ID NO:13), GCGCGATCGA GTGTTGAATA ACTCCATGTG GTAGAGTTAT TCAACACTCG ATCGCGC (SEQ ID NO:14), GCGCCACTGT CCACAGGAGA ACTCCATGTG GTAGAGTTCT CCTGTGGACA GTGGGCG (SEQ ID NO:15), and GCGCACAGTA CCCTGGAGAA ACTCCATGTG GTAGAGTTTC TCCAGGGTAC TGTGCGC (SEQ ID NO:16); and/or and shmiR molecule having the sequence of: TGTTTGAATG AGGCTTCAGT ACTTTACAGA ATCGTTGCCT GCACATCTTG GAAACACTTG CTGGGATTAC TTCGACTTCT TAACCCAACA GAAGGCTCGA GAAGGTATAT TGCTGTTGAC AGTGAGCGGC GCGATCGAGT GTTGAATAAT AGTGAAGCCA CAGATGTATT ATTCAACACT CGATCGCGCT GCCTACTGCC TCGGACTTCA AGGGGCTAGA ATTCGAGCAA TTATCTTGTT TACTAAAACT GAATACCTTG CTATCTCTTT GATACATTTT TACAAAGCTG AATTAAAATG GTATAAATTA AATCACT (SEQ ID NO:17, nucleotides 1-128=miR-E backbone, nucleotides 129-149=passenger sequence, nucleotides 150-168=loop sequence, nucleotides 169-189=guide sequence, and nucleotides 190-317=miR-E backbone). See Moffat, et al. (2006) Cell 124:1283; Guda, et al. (2015) Mol. Ther. 23(9):1465-74; and U.S. Pat. No. 9,228,185. By “inhibits BCL11A expression” is meant that the amount of expression of BCL11A is at least 5% lower in populations treated with a BCL11A inhibitory RNA, than a comparable, control population, wherein no BCL11A inhibitory RNA is present. It is preferred that the percentage of BCL11A expression in a BCL11A inhibitory RNA-treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no BCL11A inhibitory RNA is added.

In certain embodiments, a DNA construct harboring an erythroid lineage-specific promoter from glycophorin A (and optionally an enhancer) is used to treat, prevent, or ameliorate of a number of disorders extending well beyond the hemoglobinopathies. Red blood cell precursors are a useful cell population in which to express polypeptides that can be secreted into the circulation and thus delivered systemically. An example of such in vivo protein delivery is human Factor IX, a clotting factor that is missing in patients with Hemophilia B, see, e.g., Chang, et al. (2008) Mol. Therapy 16:1745-1752.

In other embodiments, cells transduced with a DNA construct of the invention can be used as “factories” for protein secretion, in vitro, ex vivo, or in vivo. For example, a DNA construct harboring an erythroid lineage-specific promoter from glycophorin A can be used for large-scale in vitro production of proteins from erythroid cells differentiated from HSCs or from embryonic stem cells. Exemplary proteins that could be expressed in this way include, but are not limited to, adenosine deaminase, the enzymes affected in lysosomal storage diseases, apolipoprotein E, brain-derived neurotropihic factor (BDNF), bone morphogenetic protein 2 (BMP-2), bone morphogenetic protein 6 (BMP-6), bone morphogenetic protein 7 (BMP-7), cardiotrophin 1 (CT-1), CD22, CD40, ciliary neurotrophic factor (CNTF), CCL1-CCL28, CXCL1-CXCL17, CXCL1, CXCL2, CX3CL1, vascular endothelial cell growth factor (VEGF), erythropoietin, Factor IX, Factor VIII, epidermal growth factor (EGF), FAS-ligand, fibroblast growth factor 1 (FGF-1), fibroblast growth factor 2 (FGF-2), fibroblast growth factor 4 (FGF-4), fibroblast growth factor 5 (FGF-5), fibroblast growth factor 6 (FGF-6), fibroblast growth factor 7 (FGF-7), fibroblast growth factor 10 (FGF-10), Flt-3, granulocyte colony-stimulating factor (G-CSF), granulocyte macrophage stimulating factor (GM-CSF), growth hormone, hepatocyte growth factor (HGF), interferon alpha (IFN-α), interferon beta (IFN-β), interferon gamma (IFN-γ), insulin, glucagon, insulin-like growth factor 1 (IGF-1), insulin-like growth factor 2 (IGF-2), interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 15 (IL-15), interleukin 17 (IL-17), interleukin 19 (IL-19), macrophage colony-stimulating factor (M-CSF), monocyte chemotactic protein 1 (MCP-1), macrophage inflammatory protein 3a (MIP-3a), macrophage inflammatory protein 3b (MIP-3b), nerve growth factor (NGF), neurotrophin 3 (NT-3), neurotrophin 4 (NT-4), parathyroid hormone, platelet-derived growth factor AA (PDGF-AA), platelet-derived growth factor AB (PDGF-AB), platelet-derived growth factor BB (PDGF-BB), platelet-derived growth factor CC (PDGF-CC), platelet-derived growth factor DD (PDGF-DD), RANTES, stem cell factor (SCF), stromal cell-derived factor 1 (SDF-1), transforming growth factor alpha (TGF-α), transforming growth factor beta (TGF-β), tumor necrosis factor alpha (TNF-α), Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16, Sonic hedgehog, Desert hedgehog, and Indian hedgehog.

Retroviral and lentiviral vectors have been tested and found to be suitable delivery vehicles for the stable introduction of genes of interest into the genome of a broad range of target cells. The present invention contemplates, in part, gene therapy vectors that can be used to deliver one or more nucleotide coding sequences to a cell to increase expression of the polypeptide encoded by the coding sequence in the cell. While the skilled artisan will appreciate that such gene therapy vectors may be produced using a variety of different viral vectors, in particular embodiments, the gene therapy vector is a retroviral vector or a lentiviral vector, in part since lentiviral vectors are capable of providing efficient delivery, integration and long-term expression of transgenes into non-dividing cells both in vitro and in vivo. A variety of lentiviral vectors are known in the art. See, e.g., U.S. Pat. Nos. 6,013,516 and 5,994,136, which may be adapted to produce a gene therapy vector of the present invention. In general, vectors of use in this invention are plasmid-based or virus-based, and are configured to carry the essential sequences for transfer of a nucleotide coding sequence into a host cell.

In illustrative embodiments, the lentiviral vector is an HIV vector. Thus, the vectors may be derived from human immunodeficiency-1 (HIV-1), human immunodeficiency-2 (HIV-2), simian virus immunodeficiency (SIV), feline immunodeficiency virus (FIV), bovine immunodeficiency virus (BIV), Jembrana Disease Virus (JDV), equine infectious anemia virus (EIAV), caprine arthritis encephalitis virus (CAEV) and the like. HIV-based vector backbones (i.e., HIV cis-acting sequence elements and HIV gag, pol and rev genes) are generally preferred in connection with most aspects of the present invention in that HIV-based constructs are the most efficient at transduction of human cells.

In one embodiment, the invention provides a gene therapy vector having one or more long terminal repeats (LTRs) and an erythroid lineage-specific promoter from a glycophorin A gene (optionally including an enhancer) operably linked to a nucleotide coding sequence of interest. In various embodiments, the design of the vector will be made with the goal of treating, preventing, or ameliorating a particular hematopoietic disease, disorder, or condition. For example, the present invention contemplates vectors for gene therapy of hemoglobinopathies that include a nucleotide coding sequence of interest selected from the group of: human α-globin, human β-globin, human δ-globin, and human γ-globin, or biologically variants or fragments thereof. Alternatively, or in addition to, the present invention provides vectors for gene therapy of hemoglobinopathies that include an erythroid lineage-specific promoter (e.g., a glycophorin A, Ankyrin, beta-Spectrin, or Adducine 2 promoter, optionally including an erythroid lineage-specific enhancer) operably linked to a nucleotide sequence encoding an inhibitory RNA (e.g., siRNA or shRNA) that inhibits the expression of BCL11A.

The Ankyrin promoter sequence is known in the art and includes an approximately 270-300 bp region 5′ of the ANK-1 gene as described by Sabatino, et al. (2000) J. Biol. Chem. 275:28549-54; Moreau-Gaudry et al. (2001) Blood 98:2664-72 and Gallagher, et al. (2000) Blood 96:1136-43. The beta-Spectrin promoter is also known in the art and includes an approximately 576 bp region 5′ of the β-spectrin coding sequence as described by Sabatino, et al. (1998) Mol. Cell Biol. 18:6634-40 and Gallagher, et al. (1999) J. Biol. Chem. 274:6062-73. Likewise the Adducine 2 promoter is known in the art and described by Yenerel, et al. (2005) Exp Hematol. 33:758-66.

Exemplary gene therapy vectors of this invention include a GATA-1 enhancer (e.g., SEQ ID NO:4) and glycophorin A promoter (e.g., SEQ ID NO:1 or SEQ ID NO:2) operably linked to a nucleic acid molecule encoding an artificial zinc finger protein or globin; a BCL11A+58 enhancer (e.g., SEQ ID NO:3) and glycophorin A promoter operably linked to a nucleic acid molecule encoding an artificial zinc finger protein or globin; a HS40 enhancer (e.g., SEQ ID NO:5) and glycophorin A promoter operably linked to a nucleic acid molecule encoding an artificial zinc finger protein or globin; a GATA-1 enhancer and glycophorin A promoter operably linked to a nucleic acid molecule encoding an shRNA targeting BCL11A (e.g., SEQ ID NO:10 or SEQ ID NO:11); a BCL11A+58 enhancer and glycophorin A promoter operably linked to a nucleic acid molecule encoding an shRNA targeting BCL11A; a HS40 enhancer and glycophorin A promoter operably linked to a nucleic acid molecule encoding an shRNA targeting BCL11A; a GATA-1 enhancer and Ankyrin promoter operably linked to a nucleic acid molecule encoding an shRNA targeting BCL11A; a BCL11A+58 enhancer and Ankyrin promoter operably linked to a nucleic acid molecule encoding an shRNA targeting BCL11A; a HS40 enhancer and Ankyrin promoter operably linked to a nucleic acid molecule encoding an shRNA targeting BCL11A; a GATA-1 enhancer and beta-Spectrin promoter operably linked to a nucleic acid molecule encoding an shRNA targeting BCL11A; a BCL11A+58 enhancer and beta-Spectrin promoter operably linked to a nucleic acid molecule encoding an shRNA targeting BCL11A; a HS40 enhancer and beta-Spectrin promoter operably linked to a nucleic acid molecule encoding an shRNA targeting BCL11A; a GATA-1 enhancer and Adducine 2 promoter operably linked to a nucleic acid molecule encoding an shRNA targeting BCL11A; a BCL11A+58 enhancer and Adducine 2 promoter operably linked to a nucleic acid molecule encoding an shRNA targeting BCL11A; and a HS40 enhancer and Adducine 2 promoter operably linked to a nucleic acid molecule encoding an shRNA targeting BCL11A. In certain embodiments, the gene therapy vector is a lentiviral vector harboring a BCL11A+58 enhancer of SEQ ID NO:3 and glycophorin A promoter of SEQ ID NO:1 or SEQ ID NO:2 operably linked to a nucleic acid molecule encoding an shRNA targeting BCL11A (e.g., SEQ ID NO:10 or SEQ ID NO:11). In other embodiments, the gene therapy vector is a lentiviral vector harboring a BCL11A+58 enhancer of SEQ ID NO:3 and Ankyrin promoter operably linked to a nucleic acid molecule encoding an shRNA targeting BCL11A (e.g., SEQ ID NO:10 or SEQ ID NO:11).

The present invention further includes pharmaceutical compositions containing one or more DNA constructs or gene therapy vectors of the invention or transduced cells containing the same and a pharmaceutically acceptable carrier. As used herein “pharmaceutically acceptable carrier” includes, but is not limited to, a solvent, dispersion medium, coating, antibacterial or antifungal agent, isotonic or absorption delaying agent, and the like that are physiologically compatible, including pharmaceutically acceptable cell culture medium. In one embodiment, a composition containing a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well-known in the art. Except insofar as any conventional media or agent is incompatible with the DNA construct or transduced cells, use thereof in the pharmaceutical compositions of the invention is contemplated.

The compositions of the invention may include one or more DNA constructs, vectors comprising the same, transduced cells, etc., herein, formulated in as described pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., cytokines, growth factors, hormones, small molecules or various pharmaceutically-active agents. Any other agents may be included in the compositions, provided that the additional agents do not adversely affect the ability of the composition to deliver the intended gene therapy.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens. In certain circumstances it will be desirable to deliver the compositions disclosed herein parenterally, intravenously, intramuscularly, or even intraperitoneally as described, for example, in U.S. Pat. Nos. 5,543,158, 5,641,515 and 5,399,363. The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, MD: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards.

In certain embodiments, the delivery may occur by use of liposomes, nanocapsules, microparticles, microspheres, lipid particles, or vesicles for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

It will also be understood that, if desired, the compositions of the invention may be administered in combination with other agents as well, such as, e.g., cells, other proteins or polypeptides or various pharmaceutically-active agents.

In certain embodiments, the present invention provides formulations or compositions suitable for the delivery of viral vector (i.e., viral-mediated transduction) including, but not limited to, retroviral (e.g., lentiviral) vectors. Exemplary formulations for ex vivo delivery may also include the use of various transfection agents known in the art, such as calcium phosphate, electoporation, heat shock and various liposome formulations (i.e., lipid-mediated transfection). Liposomes are lipid bilayers entrapping a fraction of aqueous fluid. DNA spontaneously associates to the external surface of cationic liposomes (by virtue of its charge) and these liposomes will interact with the cell membrane.

The viral vectors and DNA constructs of this invention provide improved methods of gene therapy. As used herein, the term “gene therapy” refers to the introduction of a polynucleotide into a cell's genome that restores, corrects, or modifies the gene and/or expression of the gene. In various embodiments, a viral vector of the invention includes an erythroid lineage-specific promoter from a glycophorin A gene and optionally an erythroid lineage-specific enhancer (e.g., a GATA-1, BCL11A+58 or HS40 enhancer) operably linked to a nucleotide coding sequence that provides curative, preventative, or ameliorative benefits to a subject diagnosed with or that is suspected of having monogenic disease, disorder, or condition or a disease, disorder, or condition of the hematopoietic system, e.g., a hemoglobinopathy. In other embodiments, a viral vector of the invention includes an erythroid lineage-specific promoter glycophorin A, Ankyrin, beta-Spectrin, or Adducine 2 gene and optionally an erythroid lineage-specific enhancer (e.g., a GATA-1, BCL11A+58 or HS40 enhancer) operably linked to a BCL11A inhibitory RNA that provides curative, preventative, or ameliorative benefits to a subject diagnosed with or that is suspected of a hemoglobinopathy. Accordingly, this invention also provides a method for preventing or treating a hematopoietic disorder by administering to a subject in need of treatment an effective amount of a DNA construct or viral vector disclosed herein. In certain embodiments, the hematopoietic disorder is a hemoglobinopathy. In one embodiment, the DNA construct includes an erythroid lineage-specific promoter from a glycophorin A gene and an erythroid lineage-specific enhancer (e.g., a GATA-1, BCL11A+58 or HS40 enhancer) operably linked to a nucleotide sequence encoding an artificial zinc finger protein. In another embodiment, the DNA construct includes an erythroid lineage-specific promoter from a glycophorin A, Ankyrin, beta-Spectrin, or Adducine 2 gene and an erythroid lineage-specific enhancer (e.g., a GATA-1, BCL11A+58 or HS40 enhancer) operably linked to a nucleotide sequence encoding a BCL11A inhibitory RNA (e.g., an siRNA or shRNA).

As used herein, the term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins is present in the blood (e.g., sickle cell/Hb-C disease).

The term “sickle cell anemia” or “sickle cell disease” is defined herein to include any symptomatic anemic condition which results from sickling of red blood cells. Manifestations of sickle cell disease include, e.g., anemia, pain, and/or organ dysfunction such as renal failure, retinopathy, acute-chest syndrome, ischemia, priapism and stroke. Also included in the term “sickle cell disease” are acute episodes of musculoskeletal pain, which affect primarily the lumbar spine, abdomen, and femoral shaft, and which are similar in mechanism and in severity to the bends. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia and dehydration, all of which potentiate intracellular polymerization of HbS.

As used herein, the term “thalassemia” encompasses hereditary anemias that occur due to mutations affecting the synthesis of hemoglobin. Thus, the term includes any symptomatic anemia resulting from thalassemic conditions such as severe or β thalassemia, thalassemia major, thalassemia intermedia, and thalassemias such as hemoglobin H disease. A thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles.

In various embodiments, a viral vector (e.g., a retroviral vector) is administered by direct injection to a cell, tissue, or organ of a subject in need of gene therapy, in vivo. In other embodiments, cells are transduced in vitro or ex vivo with vectors of the invention, and optionally expanded ex vivo. The transduced cells are then administered to a subject in need of gene therapy.

Cells suitable for transduction and administration in the gene therapy methods of the invention include, but are not limited to stem cells, progenitor cells, and differentiated cells. In certain embodiments, the transduced cells are embryonic stem cells, bone marrow stem cells, umbilical cord stem cells, placental stem cells, mesenchymal stem cells, neural stem cells, liver stem cells, pancreatic stem cells, cardiac stem cells, kidney stem cells, hematopoietic stem cells.

In various embodiments, the use of stem cells is preferred because they have the ability to differentiate into the appropriate cell types when administered to a particular biological niche, in vivo. The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Asymmetric cell division does not increase the number of cells. Symmetric cell division produces two identical daughter cells.

As used herein, the term “pluripotent” means the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. By comparison, the term “multipotent” refers to the ability of an adult stem cell to form multiple cell types of one lineage. For example, hematopoietic stem cells are capable of forming all cells of the blood cell lineage, e.g., lymphoid and myeloid cells. The term “progenitor” or “progenitor cell” refers to a cell that has the capacity to self-renew and to differentiate into more mature cells. Progenitor cells have a reduced potency compared to pluripotent and multipotent stem cells. Many progenitor cells differentiate along a single lineage, but may also have quite extensive proliferative capacity.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to a multipotent stem cell that gives rise to all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See U.S. Pat. Nos. 5,635,387, 5,460,964, 5,677,136, 5,750,397, 5,759,793, 5,681,599, and 5,716,827). When transplanted into lethally irradiated animals or humans, hematopoietic stem and progenitor cells can repopulate the erythroid, neutrophil-macrophage, megakaryocyte and lymphoid hematopoietic cell pool. In preferred embodiments, the transduced cells are hematopoietic stem and/or progenitor cells isolated from bone marrow, umbilical cord blood, or peripheral circulation. In particular preferred embodiments, the transduced cells are hematopoietic stem cells isolated from bone marrow, umbilical cord blood, or peripheral circulation.

HSCs may be identified according to certain phenotypic or genotypic markers. For example, HSCs may be identified by their small size, lack of lineage (lin) markers, low staining (side population) with vital dyes such as rhodamine 123 or Hoechst 33342, and presence of various antigenic markers on their surface, many of which belong to the cluster of differentiation series (e.g., CD34, CD38, CD90, CD133, CD105, CD45, and c-kit, the receptor for stem cell factor). HSCs are mainly negative for the markers that are typically used to detect lineage commitment, and, thus, are often referred to as Lin(−) cells.

A “subject,” as used herein, includes any animal that exhibits a symptom of a monogenic disease, disorder, or condition that can be treated with the gene therapy vectors, cell-based therapeutics, and methods disclosed elsewhere herein. In preferred embodiments, a subject includes any animal that exhibits symptoms of a disease, disorder, or condition of the hematopoietic system, e.g., a hemoglobinopathy, that can be treated with the gene therapy vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

Typical subjects include animals that exhibit aberrant amounts (lower or higher amounts than a “normal” or “healthy” subject) of one or more physiological activities that can be modulated by gene therapy. As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

Without wishing to be bound to any particular theory, an important advantage provided by the vectors, compositions, and methods of the present invention is the high efficacy of gene therapy that can be achieved by administering populations of expressing high a cells levels of protein/inhibitory RNA of interest. The transduced cells may be administered as part of a bone marrow or cord blood transplant in an individual that has or has not undergone bone marrow ablative therapy. In one embodiment, transduced cells of the invention are administered in a bone marrow transplant to an individual that has undergone chemoablative or radioablative bone marrow therapy. It is contemplated that the combination of promoters and enhancers described herein can provide a substantial increase in expression of, e.g., shRNA and hence more effective knock-down of BCL11A, without increased non-erythroid expression of shRNA.

The following non-limiting examples are provided to further illustrate the present invention.

### EXAMPLE 1: DEVELOPMENT OF A LENTIVIRAL GENE THERAPY VECTOR FOR THE TREATMENT OF HEMOGLOBINOPATHIES

Media. Expansion media was composed of 2 mM L-Ala-L-Glu, 50 U/ml penicillin, 50 μg/ml streptomycin, 2 IU/mL EPO, 20% heat-inactivated FBS, 20 ng/ML hSCF and 1 ng/ml hIL-3. Differentiation media was composed of 2 mM L-Ala-L-Glu, 50 U/ml penicillin, 50 μg/ml streptomycin, 2 IU/mL EPO, 20% heat-inactivated FBS, and 200 μg/ml apo-transferrin.

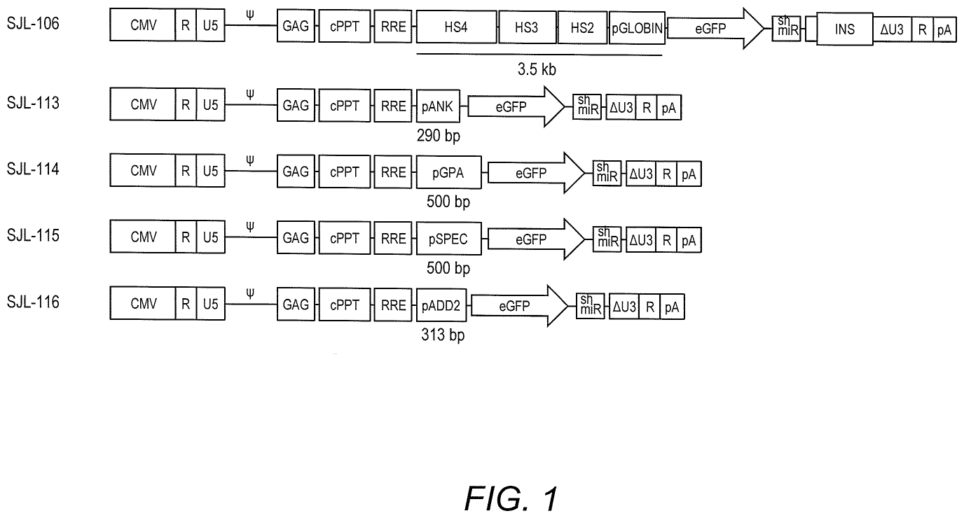

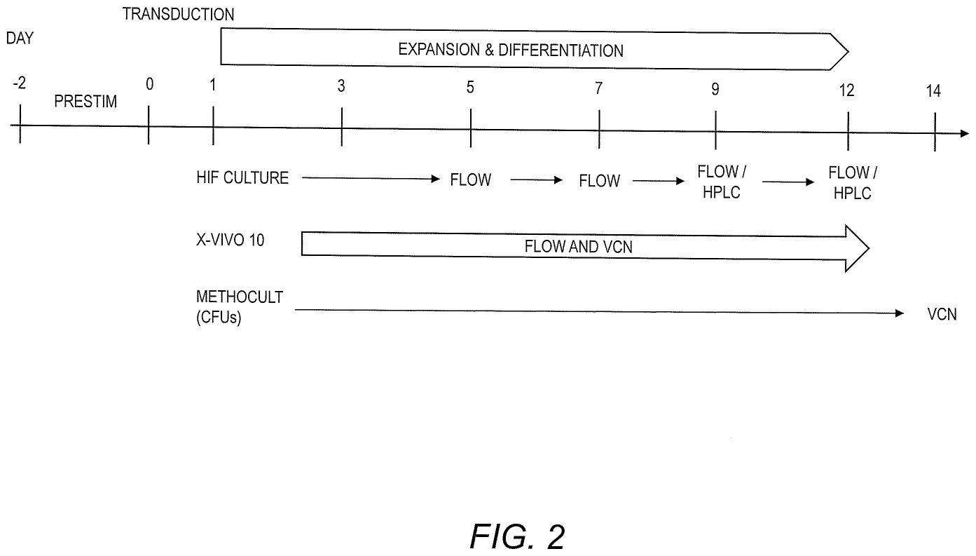

Results. To identify an erythroid-specific promoter that induces elevated γ-globin expression, lentiviral vectors containing the LCR β-globin, Ankyrin, Glycophorin A, Spectrin, or Adducin2 promoter regions were generated with eGFP and shRNA against BCL11A in miR backbone (shmiR) as a reporter (FIG. 1). As shown in FIG. 2, an in vitro erythroid differentiation culture was used to assess promoter activity. Cells were transduced with the lentiviral vectors described above, expanded (days 1-7), and differentiated (days 7-12). EGFP expression was monitored over time by flow cytometry for X-VIVO (hematopoietic media) cultures, which represents background expression in HSPC (Table 1), and HiF erythroid differentiation cultures (Table 2). The ideal erythroid specific promoter gives low eGFP expression in X-VIVO culture as in Table 1 but high expression in HiF erythroid differentiation culture as in Table 2.

TABLE 1

| | % Expression, Days Post-Transduction | | | | |
|---|---|---|---|---|---|
| Vector | D2 | D5 | D7 | D9 | D12 |
| Mock | 0 | 0 | 0 | 0 | 0 |
| GFP | 65.1 | 66.3 | 67.9 | 65.2 | 62.8 |
| SJL-106 | 29.5 | 28.3 | 29.1 | 25.6 | 19.1 |
| (pGlobin) | | | | | |
| SJL-113 | 31.4 | 42.1 | 48.1 | 42.1 | 40.1 |
| (pAnk) | | | | | |
| SJL-114 | 20.3 | 15.9 | 12.4 | 8.9 | 6.4 |
| (pGpA) | | | | | |
| SJL-115 | 35.7 | 48.9 | 60.3 | 59.6 | 66 |
| (pSpec) | | | | | |
| SJL-116 | 56.0 | 59.2 | 67.6 | 56.8 | 50.8 |
| (pAdd2) | | | | | |

TABLE 2

| | % Expression, Days Post-Transduction | | | | |
|---|---|---|---|---|---|
| Vector | D2 | D5 | D7 | D9 | D12 |
| Mock | 0 | 0 | 0 | 0 | 0 |
| GFP | 64.5 | 76.1 | 81.3 | 84.3 | 77.6 |
| SJL-106 | 20.5 | 48.6 | 55.1 | 50.4 | 33.6 |
| (pGlobin) | | | | | |
| SJL-113 | 20.5 | 52.4 | 76.6 | 89.2 | 92.8 |
| (pAnk) | | | | | |
| SJL-114 | 17.2 | 40.4 | 62.9 | 75.2 | 59.8 |
| (pGpA) | | | | | |
| SJL-115 | 28.4 | 52.7 | 69.9 | 85.6 | 84.7 |
| (pSpec) | | | | | |
| SJL-116 | 38.4 | 61.1 | 78.3 | 88.2 | 90.7 |
| (pAdd2) | | | | | |

In addition, HPLC was used as a robust readout to assess HbF & HbA and γ-globin chain (Reversed-phase chromatography) expression levels at day 9 and day 12 of HiF erythroid differentiation cultures (Table 3).

TABLE 3

| | % HbF/Total Hb_{IE-HPLC} | |
|---|---|---|
| Vector | D9 | D12 |
| Mock | 11.0 | 7.4 |
| MSCV-GFP | 11.6 | 7.6 |
| SJL-106 | 34.8 | 32.5 |
| (pGlobin-D12) | | |
| SJL-108 | 21.4 | 17.3 |
| (pGlobin-E51) | | |
| SJL-113 | 56.2 | 62.3 |
| (pAnk) | | |
| SJL-114 | 45.2 | 45.7 |
| (pGpA) | | |
| SJL-115 | 44.3 | 49.6 |
| (pSpec) | | |
| SJL-116 | 61.9 | 68.5 |
| (pAdd2) | | |

What is claimed is:

1. A DNA construct for increased erythroid lineage-specific expression of a nucleotide coding sequence comprising:
a erythroid lineage-specific promoter comprising a nucleotide sequence having at least 90% sequence identity to SEQ ID NO:1 or SEQ ID NO:2;
a nucleotide coding sequence operably linked to the promoter sequence; and
a BCL11A+58 enhancer for increasing erythroid lineage-specific expression of the nucleotide coding sequence as compared to a DNA construct not comprising the BCL11A+58 enhancer, wherein the BCL11A+58 enhancer comprises the nucleotide sequence of SEQ ID NO:3 and is operably linked upstream of said erythroid lineage-specific promoter,
wherein the nucleotide coding sequence encodes a RNA or protein and does not encode glycophorin A.

2. The DNA construct of claim 1, wherein the RNA is a BCL11A inhibitory RNA comprising a nucleotide sequence selected from the group consisting of SEQ ID NO:6-17.

3. The DNA construct of claim 1, wherein the protein is an artificial zinc finger protein or globin.

4. The DNA construct of claim 1, wherein said construct comprises a gene therapy vector.

5. The DNA construct of claim 4, wherein the gene therapy vector is a viral vector.

6. A method for preventing or treating a hematopoietic disorder comprising administering to a subject in need of treatment an effective amount of the DNA construct of claim 5 thereby treating the subject's hematopoietic disorder.

7. The method of claim 6, wherein the hematopoietic disorder is a hemoglobinopathy.


SEQUENCE LISTING (17 sequences)

SEQ ID NO: 1 - DNA, 407 bp, Homo sapiens
  source 1..407: mol_type = genomic DNA
ttgcatctcatgtctcttacattgctgtgtggctcaccatgagtttgggagtctttcagaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcattt

SEQ ID NO: 2 - DNA, 502 bp, Homo sapiens
  source 1..502: mol_type = genomic DNA
ttgcatctcatgtctcttacattgctgtgtggctcaccatgagtttgggagtctttcagaacctcagaacactcaaatgatttaaatttctcaaatacattcatttcacatataggaagtcactttcatttggaccactgggtcttgacattagaaatgagaaggtccatggctccacaacagctacctcagcctggcacgtgccctggcctcagagattcacagtccagttctttgtccagttgggtggctcctgtctaccaccttaccatgcccacttaactgatgcaaagttaatatcacaagtagcaacctgttccttgcagtgaaaattttacttaccactttcatagccccaagatatccatgtatctttattaacaggcgcttaacaacttgcatcatttaaaatgcctcccctgcctatcagctgatgatggccgcaggaaggtgggcctggaagataacagctagcaggctaaggtcagacactgacacttgc

SEQ ID NO: 3 - DNA, 158 bp, Homo sapiens
  source 1..158: mol_type = genomic DNA
gaggcccccctgggcaaacggccaccgatggagaggtctgccagtcctcttctaccccacccacgcccccaccctaatcagaggccaaacccttcctggagcctgtgataaaagcaactgttagcttgcactagactagcttcaaagttgtattgacc

SEQ ID NO: 4 - DNA, 213 bp, Homo sapiens
  source 1..213: mol_type = genomic DNA
cctcgaggaatcatccctggctcccacctcagtttcccgcctccaaggcagcatggcgggcaagaagttgaggccactgtccctgggtgttcctacccccacaccctcaccccaagacagcctgttactgcggcgccaacagccacggtcgcctacatctgataagacttatctgctgccccagggcaggccggagctggcgtaagccccagt

SEQ ID NO: 5 - DNA, 255 bp, Homo sapiens
  source 1..255: mol_type = genomic DNA
tctggaacctatcagggaccacagtcagccaggcaagcacatctgcccaagccaagggtggaggcatgcagctgtgggggtctgtgaaaacacttgagggagcagataactgggccaaccatgactcagtgcttctggaggccaacaggacttctgagtcatcctgtgggggtggaggtgggacaagggaaaggggtgaatggtactgctgattacaacctctggtgctgcctccccctcctgtttatctgagag

SEQ ID NO: 6 - RNA, 19 nt, synthetic construct
  misc_feature 1..19: note = Synthetic oligonucleotide
  source 1..19: mol_type = other RNA
gagcacaaacggaaacaat

SEQ ID NO: 7 - RNA, 19 nt, synthetic construct
  misc_feature 1..19: note = Synthetic oligonucleotide
  source 1..19: mol_type = other RNA
gccacaggatgacgattgt

SEQ ID NO: 8 - RNA, 18 nt, synthetic construct
  misc_feature 1..18: note = Synthetic oligonucleotide
  source 1..18: mol_type = other RNA
gcacttaagcaaacggga

SEQ ID NO: 9 - RNA, 19 nt, synthetic construct
  misc_feature 1..19: note = Synthetic oligonucleotide
  source 1..19: mol_type = other RNA
acagaacactcatggatta

SEQ ID NO: 10 - DNA, 56 bp, synthetic construct
  misc_feature 1..56: note = Synthetic oligonucleotide
  source 1..56: mol_type = other DNA
ccggcgcacagaacactcatggattctcgagaatccatgagtgttctgtgcgtttt

SEQ ID NO: 11 - DNA, 58 bp, synthetic construct
  misc_feature 1..58: note = Synthetic oligonucleotide
  source 1..58: mol_type = other DNA
ccggccagaggatgacgattgtttactcgagtaaacaatcgtcatcctctggtttttg

SEQ ID NO: 12 - DNA, 57 bp, synthetic construct
  misc_feature 1..57: note = Synthetic oligonucleotide
  source 1..57: mol_type = other DNA
gcgctcggagactccagacaactccatgtggtagagttgtctggagtctccgagcgc

SEQ ID NO: 13 - DNA, 57 bp, synthetic construct
  misc_feature 1..57: note = Synthetic oligonucleotide
  source 1..57: mol_type = other DNA
gcgcttctcttgcaacacgcactccatgtggtagagtgcgtgttgcaagagaagcgc

SEQ ID NO: 14 - DNA, 57 bp, synthetic construct
  misc_feature 1..57: note = Synthetic oligonucleotide
  source 1..57: mol_type = other DNA
gcgcgatcgagtgttgaataactccatgtggtagagttattcaacactcgatcgcgc

SEQ ID NO: 15 - DNA, 57 bp, synthetic construct
  misc_feature 1..57: note = Synthetic oligonucleotide
  source 1..57: mol_type = other DNA
gcgccactgtccacaggagaactccatgtggtagagttctcctgtggacagtgggcg

SEQ ID NO: 16 - DNA, 57 bp, synthetic construct
  misc_feature 1..57: note = Synthetic oligonucleotide
  source 1..57: mol_type = other DNA
gcgcacagtaccctggagaaactccatgtggtagagtttctccagggtactgtgcgc

SEQ ID NO: 17 - DNA, 317 bp, synthetic construct
  misc_feature 1..317: note = Synthetic oligonucleotide
  source 1..317: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcggcgcgatcgagtgttgaataatagtgaagccacagatgtattattcaacactcgatcgcgctgcctactgcctcggacttcaaggggctagaattcgagcaattatcttgtttactaaaactgaataccttgctatctctttgatacatttttacaaagctgaattaaaatggtataaattaaatcact